(19) United States
(12) Patent Application Publication
(10) Pub. No.: US 2002/0012648 A1
(43) Pub. Date: Jan. 31, 2002

(54) High phospholipid-containing dermatological compositions

(76) Inventor: Frank T. Orthoefer, Chesterfield, MO (US)

Correspondence Address: Robert M. Schulman, Esq., Hunton & Williams, 1900 K Street, NW, Suite 1200, Washington, DC, 20006, US

(21) Appl. No.: 09/852,286

(22) Filed: May 10, 2001

Related U.S. Application Data
(63) Continuation-in-part of application No. 09/245,289, filed on Feb. 5, 1999, now Pat. No. 6,312,703.
(60) Provisional application No. 60/073,990, filed on Feb. 6, 1998.

(51) Int. Cl. A61K 7/075; A61K 7/08; A61K 9/127

(52) U.S. Cl. 424/70.27; 424/70.28; 424/70.23

(57) ABSTRACT

A composition is described for cosmetic and/or pharmaceutical treatment of the outermost layers of the epidermis by topical application to the skin. The preparation comprises a relatively high amount of phospholipids that applies a thin monomolecular layer to the skin, permitting natural hydration. The chemical composition of the phospholipids provides antioxidant protection and natural sun-blocking effects. All natural oils are incorporated into the phospholipid phase. Since no synthetic emulsifiers or harsh alcohols are used in the composition, it is non-irritating to the skin. Moreover, the cosmetics of the present invention exhibit both good emulsion stability and storage stability while providing a good skin feel.

This application is a continuation-in-part of pending U.S. patent application Ser. No. 09/245,289, filed Feb. 5, 1999, which claims the benefit of U.S. Provisional Application Serial No. 60/073,990, filed Feb. 6, 1998, all of which are incorporated herein by reference. This application is also related to U.S. patent application Ser. No. <Not Yet Assigned>, entitled “Liquid-Solid Form Lecithin Compositions” and corresponding to Attorney Docket No. 58781.000015.

### FIELD OF THE INVENTION

The present invention relates to skin care compositions comprised of phospholipids (also known as lecithin), or enriched phospholipids, as active agents for the skin in an amount of at least 10% up to 40% by weight of the total cosmetic composition. The present invention also relates to the method of making the cosmetic compositions. The cosmetics produced by the present invention have high stability, provide good skin feel, and permit natural hydration of the skin.

### BACKGROUND OF THE INVENTION

There are many forms of lecithin, that are composed of phospholipids, which are known in the art. Typical lecithins comprise deoiled granules or powders having approximately the following composition (Orthoefer, F., Lecithin and Health, Vital Health Pub, Bloomingdale, Ill., p.21.):

| | |
|---|---|
| Phosphatidylcholine | 23% |
| Phosphatidylethanolamine | 21% |
| Phosphatidylinositol | 19% |
| Other phospholipids | 15% |
| Phosphatidic acid | 6% |
| Glycolipids | 14% |

In addition, lecithins also comprise enriched phospholipid compounds in which, for example, the phosphatidylcholine content exceeds 23%, or the phosphatidylinositol content exceeds 19%, or the phosphatidyiethanolamine content exceeds 21%.

Most phospholipids are amphiphilic, having a hydrophilic polar component and a lipophilic non-polar component. Therefore, phospholipids tend to form a monomolecular film when suspended in a water/oil environment, with the polar component towards the water and the non-polar components away from the water. In water solutions, phospholipids prefer to form spherical bilayers known in the trade as vesicles, nanoparticles, liposomes, or lamellar layers. In such bilayer vesicles, no non-polar component of the phospholipid has any contact with the water solution.

Lecithins and other phospholipids have been used in the pharmaceutical industry to formulate carriers for water-insoluble drugs. U.S. Pat. No. 5,173,303 to Lau et al. describes sequestering water-insoluble insect repellent for the skin using phospholipid lamellar layers composed of lecithin. However in addition to forming vesicles, lecithin phospholipids also can provide a soft, moisturizing feel to the skin because they have a strong affinity for the lipophilic surface of the corneum. Furthermore, these phospholipids are toxicologically safe, are antioxidants and promote shelf stability for such products. Thus it would be desirable to use phospholipids as a carrier for lipophilic ingredients for cosmetic and pharmaceutical applications.

It has been known to use a small amount of lecithin as a constituent in formulations for soaps, creams, and lotions, as well as other emulsions applied to the skin. These various emulsions containing lecithin have been used to moisturize skin and as medicated ointments for general applications, with the various emulsions and ointments rubbed onto the user's skin. In these formulations, lecithin serves the following purposes: 1) it is an emulsifier to promote solubilization of non-aqueous soluble components or medications into the aqueous phase, 2) it contributes to the stability of the emulsion, 3) it is an excellent moisturizer, 4) it delivers highly beneficial phospholipids to the skin, and 5) it contains vitamin E and polyunsaturated fats, both of which impart health benefits to the user's skin. While such emulsions and creams are useful in reducing dry skin, they can leave a greasy or sticky feeling on the user's skin. The non-aqueous portions of the known emulsions are often simply oils which do not replenish moisture or oils lost from the skin. Thus, it would be desirable to have an ointment, treatment, or emulsion, which is easy to apply and does not leave a sticky or greasy feeling on a user's skin, but which is useful in moisturizing the skin, maintaining softness, and preventing loss of water and natural oils.

Some skin care products incorporate relatively small amounts of lecithin as an emulsifying ingredient rather than as an active ingredient in itself. U.S. Pat. No. 4,424,204 to Minamino, et al. describes a skin cosmetic that consists of 18.alpha.-glycyrrhizic acid as a primary emulsifier and a water-soluble secondary emulsifier such as lecithin in an amount up to 10% by weight. In this patent, lecithin is used solely to eliminate the difficulties inherent in the use of 18.beta.-glycyrrhizin as an emulsifier alone. U.S. Pat. No. 5,922,331 to Mausner and U.S. Pat. No. 5,849,315 to Rerek, et al. describe skin care and cosmetic preparations which use lecithin solely as an emulsifier in very small amounts of between 0.5 and 1% by weight.

U.S. Pat. No. 5,520,905 to Uhlmann, et al. describes a cosmetic composition using delta-aminolevulinic acid as a sun blocker and an antioxidant up to 10% by weight, one of which may be lecithin, although it is not the preferred antioxidant. Also, Uhlmann does not acknowledge that lecithin itself is an active ingredient in multitudinous ways. The skin is composed of phospholipids and thus lecithin acts as a natural skin penetrant. Lecithin is also a UV blocker, contains vitamin E, and is a carrier for lipid soluble nutrients into the skin. Additionally, lecithin incorporates polyunsaturated fats, which provide protection to damaged skin.

Lecithin is also known to form liposomes into which active ingredients can be placed. U.S. Pat. No. 3,957,971 to Oleniacz describes a composition used to moisturize the skin, with the lecithin being used solely to create liposomes. As such, said lecithin comprises less than 1% by weight of the total composition and does not contribute as an active ingredient. U.S. Pat. No. 6,015,574 to Cannell, et al. describes a lipophilic carrier system that uses lecithin in an amount less than 5% by weight to form bilayers. Again the lecithin of Cannell is not used as an active skin care component. U.S. Pat. No. 6,203,802 to Handjani,et al. describes a cosmetic formulation comprising nanoparticle encapsulated oils. These nanoparticles are liposomes formed in the conventional manner with a small amount of lecithin forming the vesicles.

U.S. Pat. No. 4,783,450 to Fawzi et al. describes the use of lecithin as a skin penetration enhancer designed for transdermal applications, in which the described skin is mucosal and bronchial. Kato (“Effect of Egg Yolk Lecithin on Transdermal Delivery of Bunazosin Hydrochloride”, J. of Pharmacy and Pharmacology (1987 May) 39(5): 399-400) and Willimann (“Lecithin Organogel as Matrix for Transdermal Transport of Drugs”, J. of Pharmaceutical Sciences (1992 September) 81(9): 871-874) report on the effectiveness of egg lecithin and lecithin gels as efficient carriers of drugs through the skin.

Lecithin is derived from egg or soybeans and can be produced in liquid, powdered, or granular form. While lecithin has been used as an emulsifier, products based largely on phospholipids are few. U.S. patent application Ser. No. 09/243,289 describes the use of a cosmetic emollient skin bar that is composed of lecithin phospholipids from about 20% up to 100% by weight of the total composition. Other active ingredients can be cosolubilized with the lecithin to form a unique skin bar delivery system. However, the cosmetics produced by the composition of this invention are not in the form of lotions, creams or lip balms. Thus, it would be desirable to have a cosmetic lecithin composition in which one of the major active ingredients is lecithin and which could be easily applied to the skin in a liquid, semi-liquid or spreadable form.

Among the reasons most skin products have not been based on pure phospholipids are that granular and/or powdered lecithins are not easily applied to the skin, while liquid lecithins, even diluted to 50%, are viscous and not easily spreadable. Because of the unique benefits derived from using pure lecithin and enriched lecithin, it would be especially desirable to use lecithin as one of the main constituents in cosmetic formulations. It would also be desirable to have a lecithin cosmetic that incorporates dermatological preparations that would be beneficial and advantageous for the treatment of skin ailments and diseases.

### SUMMARY OF THE INVENTION

The present invention is directed to cosmetics that incorporate lecithin as an active ingredient, such as a nutraceutical agent, pharmaceutical agent, ointment, or moisturizer. In one embodiment, the liquid-solid form lecithin comprises a phospholipid having the formula C8H17O5NRR′, wherein R and R′ are fatty acids having the formula CH3(CH2)ₙCOO, with n equal to between 4 and 22. In another embodiment, the liquid-solid form lecithin comprises at least one of:

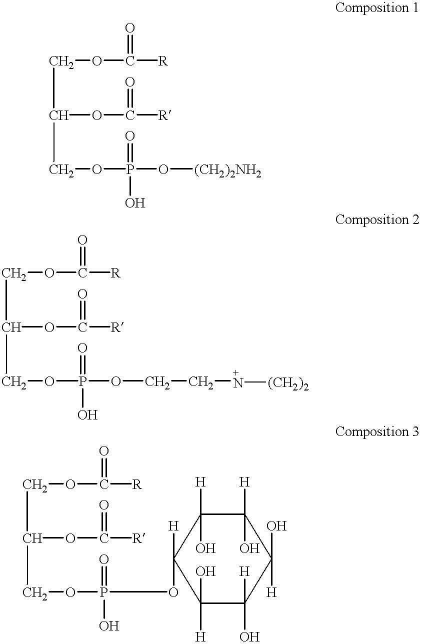

wherein R and R′ are fatty acids having the formula CH3(CH2)ₙCOOH, with n equal to between 4 and 22.

The invention also provides compositions that comprise a lecithin and can further include other active agents in addition to the lecithin. The active agent can be, for example, a nutrient, nutraceutical agent, pharmaceutical agent, fragrance, oil, moisturizer, vitamin, mineral, mW blocker, humectant, cleanser, colloidal meal, abrasive, herbal extract, phytochemical, phospholipid, or any combination thereof. These compositions can also have, for example, between 20% and 60% lecithin by weight of the total composition.

### DETAILED DESCRIPTION OF THE PREFERRED EMBODIMENTS

Phospholipids, including lecithin, have a number of desirable health benefits and are used in cosmetic products for a variety of different uses. Phospholipid compounds are high in vitamin E and fatty acids, which are beneficial when placed on the skin. Traditionally, phospholipids have been placed in cosmetics and other goods as an emulsifier rather than as an active ingredient. To date, high percentage lecithin cosmetic creams, lotions and lip protection products have not been commercially available. In addition to lecithin phospholipids as active ingredients, oils such as for example, jojoba, emu, almond, macadamia nut, borage, black current, evening primrose, high oleic sunflower, and other vegetable oils result in cosmetics that exhibit excellent emollient characteristics. Other dermatological nutrients such as the vitamins tocotrienol, tocopherol, and ascorbic acid, also can be incorporated into the lecithin cosmetic compositions and are useful in repairing damaged skin. The addition of phytochemnicals such as for example, polyphenols, sterols, and stanols, act to maintain the integrity of the skin cell membranes and to stabilize the liposomes formed when the lecithin phospholipids of the composition are hydrated.

Powdered or granular lecithins are commercially available and come in a variety of grades, including food grade, technical, bleached, unbleached, and enriched. Regardless of the type of powdered or granular phospholipids selected, it is preferred for convenience that the lecithin have a phospholipid content of at lea 90%, but not limited to this amount, and be oil free or de-oiled. Specific phospholipid components, which are available for use in the invention, include, for example, phosphatidylcholine, phosphatidylethanolamine, and phosphatidylinositol. In addition, synthetic lecithins, such as YN100 Cadberry, a synthetic phosphated diglyceride, and other synthetic phospholipids may be used. The starting lecithins can also be enriched or enhanced lecithins that are well known in the art, such as lecithins enhanced with phosphatidylcholine or phosphatides.

In one embodiment the lecithin has an acetone insoluble index equal to or greater than 90%. In another embodiment, the lecithin comprises a phospholipid having the formula C₈H₁₇O₅NRR′, wherein R and R′ are fatty acids having the formula CH₃(CH₂)ₙCOO, with n equal to between 4 and 22. In yet another embodiment, the lecithin comprises a phospholipid having a formula comprising at least one of:

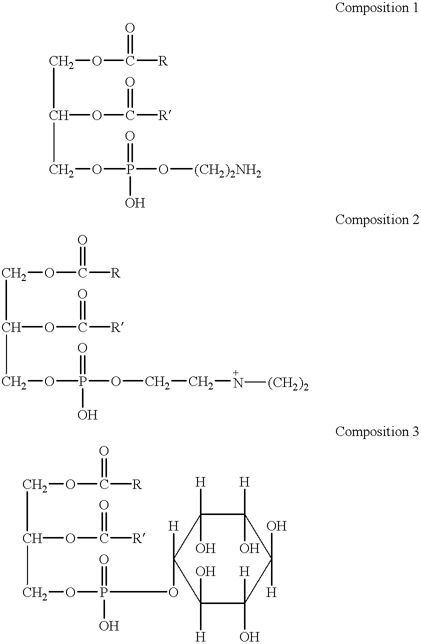

wherein R and R′ are fatty acids having the formula CH₃(CH₂)ₙCOOH, with n equal to between 4 and 22. One of skill in the art will appreciate that other fatty acids containing other R groups, including those with a cyclic structure and with or without heteroatoms, may also be used according to the present invention. The starting lecithin can also contain an amount of one or more additives, such as an active agent.

The compositions of the invention comprise lecithin as an active ingredient. In one embodiment, the lecithin comprises at least about 10% by weight of the total weight of the composition. The lecithin can comprise as much as 30% by weight of the total composition.

The compositions of the invention, which comprise lecithin as an active ingredient, are advantageous because they take advantage of the monomolecular film-forming properties of phospholipid and impart numerous health benefits to the skin of the user. Not only do phospholipids have health benefits, but they can also serve as a carrier for dermatological preparations desirable for skin health. The lecithin cosmetic may contain substances such as waxes, fatty acids, vitamins, ultraviolet light blockers, germicides, phytochemicals, oils, and additional compounds. Thus, the invention relates to lecithin cosmetic compositions that may contain additives for use on human or animal skin.

The invention also includes compositions and pharmaceutical compositions that have pro-liposomes. Several examples of the proliposome-liposome formation are known (Williams, W. P. et al., “The Pro-Liposome Method: A Practical Approach to the Problem of the Preparation and Utilization of Liposomes Suitable for Topical Applications”, in Phospholipids: Characterization, Metabolism, and Novel Biological Applications, Chapter 17, AOCS Press; Payne, N. et al., “Characterization of Proliposomes”, J. of Pharmaceutical Sciences (1986 April) 75(4):330-333; Deo, M. et al., “Proliposome based Transdermal Delivery of Levonorgestrel”, J. of Biomatter Appl. (1997 July) 12(l):77-78. In this process, the starting lecithin is mixed with one or more components (e.g., other active agents) in the presence of a small amount of water. When the resulting mixture is combined with additional water, vesicles (liposomes) are formed effectively encapsulating the active ingredients into the composition. Thus, the invention relates to lecithin cosmetic compositions that may contain other active agents, which cosmetics contain pro-liposomes that are converted to liposomes when they are hydrated.

A variety of additives can be mixed with the powdered phospholipid to form liquid-solid lecithin compositions, including fragrances, oils, moisturizers, vitamins, UV blockers, humectants, cleaning agents, colloidal meals, nutritional supplements, chelated minerals, herbal extracts, essential fatty acids, drugs, phytochemicals, amino acids, disintegrants, and combinations thereof. Which additives will be added to the lecithin cosmetic composition will depend upon the characteristics desired in the finished product. For example, a lecithin cosmetic composition designed specifically to improve psoriasis may contain tocotrienols, emu, borage, evening primrose, or omega 3 oils in addition to the phospholipids in an amount equal to at least about 20% by weight of the total lecithin cosmetic composition. A lecithin cosmetic composition designed specifically to reduce the effects of UV exposure to the skin may contain tocopherol, tannic acid, and epigallocatechin isolated from green tea.

Regardless of the ingredients incorporated into the lecithin cosmetic composition, the formulation is initiated by adding an amount of selected lecithin to the water, water-soluble agents, and polyethylene glycol at a temperature of about 65-70° C. The lipid soluble ingredients are combined together under constant mixing at between 70-80° C. The lipid soluble combination is slowly added to the aqueous mixture with constant stirring. Bactericides are then added and the material is cooled. The composition thus formed is subjected to reheating to about 70° C. at which point fragrance and any additional water is added to correct the consistency.

The following are working examples demonstrating the production and use of lecithin cosmetic compositions. It will be appreciated by someone skilled in the art that the invention is not limited to the following examples. Suppliers of the various ingredients that are cited in the said examples are:

| | |
|---|---|
| Ascorbyl Palmitate | Roche Vitamins |
| β-Carotene | Roche Vitamins |
| Borage Oil | _{—} |
| Cotton/Soy/Palm Sterine | AC Humko |
| Emu Oil | LB Processors |
| Evening Primrose Oil | _{—} |
| Germall Plus | Sutton Labs |
| Jojoba Oil | Purcell Jojoba Inc. |
| Kokam Butter | Carlisle International |
| Lecithin 5750 | Riceland Foods |
| Lecithin 6440 | Central Soya |
| Lecithin 8100 | Central Soya |
| Na Ascorbate | BASF |
| Na_Palmitate | _{—} |
| Na Stearoyl Lactylate (Emplex) | American Ingredient Co. |
| Octyl Salicylate | Rita Chemical |
| Polyethylene Glycol | Paddock Labs |
| Sterol Esters | Cognis Corp |
| Steryl Esters | Cargill |
| Tocopherol | Henkel |
| Tocotrienol | Eastman Chemical |

### EXAMPLE 1

### A42-44a

Fifty-three grams of deionized water is heated to 80° C. with 0.5 gr of polyethylene glycol. Fifteen grams of lecithin (Riceland- 5750) is slowly added with constant stirring (Phase 1). In a separate kettle the following is heated to 80° C.: 15 gr of high oleic sunflower oil, 1.5 gr each of kokam, emu oil, jojoba oil and Emplex. Add 0.60 gr of tocotrienol. Cool slightly and add 0.53 gr Germall Plus (Phase II). Slowly add the oil Phase II to the water, PEG and lecithin Phase I, stirring until cool. The resultant product is a thick cream that acts as a barrier to outside agents, and is applicable as a diaper cream or as an occlusive covering.

### EXAMPLE 2

### A42-44b

The same procedure was used as in Example 1, except that the final composition is reheated to approximately 70° C. and 50 gr of DI water is added under constant stirring (Phase III). The cosmetic is cooled. The resultant product is a lotion that is readily absorbed and leaves a thin layer of emollients on the skin.

### EXAMPLE 3

### A43-46

The same procedure was used as in Example 2 except that 15 grams of Central Soya 8100 lecithin is substituted for the Riceland 5750. Upon examination of the final product, a softer and slightly more spreadable lotion was formed.

### EXAMPLE 4

### A17-5

The same procedure was used as given in Example 1.

| Phase I: | |
|---|---|
| DI Water | 51.8% |
| Lecithin 8100 | 10.4 |
| PEG | 0.5 |
| Phase II: | |
| Hi Oleic Sunflower | 20.7 |
| Cotton/Soy Sterine | 5.3 |
| Kokam Butter | 4.1 |
| Jojoba Oil | 2.1 |
| Polysorbate | 2.1 |
| Tocopherol | 1.0 |
| Palm Sterine | 1.2 |
| Germall Plus | 0.5 |
| Fragrance | 0.3 |

This cosmetic was used at night as a repair cream for badly chapped hands and elbows

### EXAMPLE 5

### A18-7

The same procedure was used as given in Example 1.

| Phase I: | |
|---|---|
| DI Water | 23.3% |
| Lecithin 8100 | 14.2 |
| Phase II: | |
| Hi Oleic Sunflower | 40.1 |
| Cotton/Soy Sterine | 12.2 |
| Kokam Butter | 8.1 |
| Jojoba Oil | 0.4 |
| Germall Plus | 0.9 |
| Fragrance | 0.8 |

This cosmetic was used as a lip balm.

### EXAMPLE 6

### A29-21

The same procedure was used as given in Example 2.

| Phase I: | |
|---|---|
| DI Water | 42.4% |
| Lecithin 5750 | 11.9 |
| PEG | 0.4 |
| Phase II: | |
| Hi Oleic Sunflower | 11.9 |
| Cotton/Soy Sterine | 3.1 |
| Kokam Butter | 1.2 |
| Jojoba Oil | 1.2 |
| Emu Oil | 1.2 |
| Polysorbate | 1.2 |
| Tocotrienol | 0.6 |
| Steryl Esters | 3.7 |
| Palm Sterine | 0.7 |
| Germall Plus | 0.4 |
| Fragrance | 0.2 |
| Phase III | |
| DI Water | 19.9 |

This cosmetic was used as a moisturizing lotion for the body.

### EXAMPLE 7

### A17-5b

The same procedure was used as given in Example 1.

| Phase I: | |
|---|---|
| DI Water | 51.8% |
| Lecithin 8100 | 10.4 |
| PEG | 0.5 |
| Phase II: | |
| Hi Oleic Sunflower | 20.7 |
| Cotton/Soy Sterine | 5.3 |
| Evening Primrose Oil | 6.2 |
| Polysorbate | 2.1 |
| Tocopherol | 1.0 |
| Palm Sterine | 1.2 |
| Germall Plus | 0.5 |
| Fragrance | 0.3 |

This cosmetic was used at night as a repair cream for the itch associated with poison ivy.

### EXAMPLE 8

### III-24

The same procedure was used as given in Example 1.

| Phase I: | |
|---|---|
| DI Water | 50.0% |
| Lecithin 8100 | 5.0 |
| PEG | 0.9 |
| Phase II: | |
| Hi Oleic Sunflower | 25.2 |
| Cotton/Soy Sterine | 6.3 |
| Kokam Butter | 5.0 |
| Jojoba Esters | 1.3 |
| Tocopherol | 0.6 |
| Palm Sterine | 1.3 |
| Octyl Salicylate | 3.9 |
| Germall Plus | 0.5 |

This cosmetic lotion was used to protect the skin from UV radiation.

### EXAMPLE 9

### B7-83

An emollient bar was produced by the procedure used in U.S. patent application Ser. No. 09/245,289. It consisted of initially mixing together and subsequently compressing 20.6% by weight of lecithin (Riceland 5750) together with 23.1% aloe vera, 7.4% emu oil, 0.5% tocotrienol, and 0.5% ascorbic acid. In this manner, a liquid-solid form of lecithin matrix was produced with all ingredients cosolubilized with the active agent, lecithin. Ten grams of this liquid-solid form was cryogenically milled and added slowly to 10 gr of DI water at 70° C. under constant stirring. The resulting lotion was used as a skin moisturizer and a treatment against acne.

### EXAMPLE 10

A liquid-solid form cosmetic bar composition was made from phospholipid, so that the method of the invention converted the powdered or granular phospholipids into a solid phospholipid bar composition having a specific gravity of 1.2 g/ml. The cosmetic bar composition was made by adding 40 grams of powdered lecithin obtained from Archer Daniels Midland Co., having an acetone insoluble index of 98, to a die mounted on a hydraulic press. The die was purchased from VVR Scientific Products Inc located in Dallas, Tex. under catalog number 53887-003. The die had an internal diameter of 2.25 inches and an outside diameter of 3.825 inches. The hydraulic press was model number 2086, made by Carver, Inc of Dallas Tex.

After the 40 grams of powdered phospholipid were added to the die, the die was closed so that the powdered phospholipid could not escape the die. Once the die was closed, the hydraulic press was activated and 5000 psig was applied to the powdered phospholipid for 5 minutes. The pressure caused the phospholipid to form a translucent liquid-solid form phospholipid bar, similar to a bar of soap, having a specific gravity of 1.2 g/ml. The cosmetic bar made of phospholipid was then removed from the die. The bar was intact, had a diameter of 2.25 inches and was ⅜ inch tick. The cosmetic bar had non-brittle form.

The cosmetic bar made from phospholipid had an amber appearance and was a semi-transparent solid having the consistency of a soap bar. The cosmetic bar from the phospholipid was then tested on a human subject who used the cosmetic bar in the shower after washing, but in the same manner as a bar soap. After applying the cosmetic bar, the subject rinsed and dried with a towel. The subject observed a coat over his entire body surface which made the skin smooth and gave it a soft feel. The subject further observed that the coating was present for eight (8) hours and up to 12 hours later after application of the cosmetic bar to the skin.

The subject further observed that, after use, the cosmetic bar did not readily degrade and that it kept its shape after use in the shower, lasting two weeks.

### EXAMPLE 11

The same procedure was used as in Example 10 except that 45 grams of powdered phospholipid, equal to 90% by weight of the total composition, were added to the die chamber along with 5 grams of white petrolatum, equal to 10% by weight of the total composition. Compression was similar to Example 10. Upon examination of the finished product, a translucent, liquid-sold form bar was formed which was usable in the shower similar to hand soap, except it was used after rinsing, and then it too was rinsed off. After use, a thin layer of phospholipid and white petrolatum were formed on the subject's skin. It was observed that the thin film layer was especially desirable because it persisted for up to eight hours and relieved dry skin and discomfort associated with pruritis.

### EXAMPLE 12

The same procedure was used as in Example 10 except 45 grams of powdered phospholipid, equal to 90% by weight of the total composition, were added to the die chamber along with 5 grams of USP Aquaphor, equal to 10% by weight of the total composition. The phospholipid and Aquaphor were mixed in a standard food processor prior to placement in the die chamber.

The translucent, liquid-solid form bar similar to the cosmetic bar of Example 10 was formed, so that the cosmetic bar containing USP Aquaphor had the consistency of a bar soap. Again the cosmetic bar of the present example was used in a manner similar to the cosmetic bar disclosed in Example 11. The cosmetic bar of the present Example resulted in a uniform film on the skin of the user that resulted in a slight, desirable sheen to the skin surface and which provided a protective layer.

### EXAMPLE 13

The same procedure used in Example 10 was followed except 40 grams of powdered phospholipid, equal to 80% by weight of the total composition, were added to the die chamber along with 10 grams of colloidal oatmeal, equal to 20% by weight of the composition.

The resulting phospholipid and oatmeal cosmetic bar was opaque and had the consistency of a bar of soap. The phospholipid and oatmeal bar was used in a similar way as the cosmetic bar disclosed in Example 11. Use of the phospholipid and oatmeal bar resulted in the formation of a thin layer on the skin of the user. The phospholipid and oatmeal bar was particularly useful in relieving the discomfort of pruritis.

### EXAMPLE 14

A cosmetic bar similar to the bar discussed in Example 10 was prepared, but in place of a portion of phospholipid, 5 grams of lanolin was mixed with 45 grams of powdered phospholipid in a food processor. The 50 gram mixture was compressed as in Example 10.

The phospholipid lanolin bar was easily used in the shower, bath or after hand washing, especially when compared to treatment with a product as unctuous as lanolin. The bars last approximately 14 days.

### EXAMPLE 15

A cosmetic bar similar to the bar discussed in Example 10 was prepared but in place of a portion of the phospholipid, 32 grams of borage oil (Bioriginal Food and Science Corp), 0.8 grams of green tea extract (Anhui Tongling Co), and 1.6 grams of tocotrienol complex (Eastman Chemical Co), was mixed with 45.6 grams of powdered phospholipid in a food processor. The 80 gram mixture was compressed as in Example 10.

The resulting phospholipid bar was easily applied in the shower, bath, and after hand washing. This phospholipid bar and the ingredients contained therein was used as a precautionary agent against exposure to ultraviolet B radiation. The skin was soft and moist after use and the bars lasted approximately 14 days.

### EXAMPLE 16

A cosmetic bar similar to that of Example 10 was prepared, but in place of a portion of the phospholipid, 9.4 grams of evening primrose oil (Now Foods) and 2.6 grams of tocotrienol complex was mixed with 111 grams of powdered phospholipid in a food processor. The 123 gram mixture was compressed as in Example 10.

The liquid crystal evening primrose oil (EPO) bar was readily used in the shower or bath after washing and rinsing. The EPO bar was used in the treatment of plaque-type psoriasis and was effective in reducing the itching and scratching associated with that ailment. The bars lasted approximately 12 days.

### EXAMPLE 17

A cosmetic bar similar to the bar discussed in Example 10 was prepared but in place of a portion of the phospholipid, 3.2 grams of green tea extract (Anui Tonglai Corp) and 1.6 grams of tocotrienol complex (Eastman Chemical Co) were mixed with 35.2 grams of powdered phospholipid in a food processor. The 40 gram mixture was compressed as in Example 10.

The resulting phospholipid bar was easily applied in the shower, bath, and after hand washing. This phospholipid bar and the ingredients contained therein was used to reduce the itching associated with poison ivy. The skin was soft and moist after use and the bars lasted approximately 14 days.

### EXAMPLE 18

A skin bar was made using the following ingredients: 14.2 % Sodium Ascorbate (BASF), 82.8% lecithin (Riceland 5730) and 3% water. After mixing and compressing at 3500 psi for 4 minutes, the ingredients were cosolubilized. The bar was used to prevent oxidative damage to the skin.

We claim:

1. A dermatological composition comprising a lecithin component and an additive component, said lecithin component comprising between about 10% and about 30% by weight of the total weight of the dermatological composition, said lecithin component having an acetone insoluble content equal to at least about 90% and a density from about 0.93 to about 1.2.

2. The dermatological composition of claim 1, wherein the lecithin component has a phospholipid content of at least about 90%.

3. The dermatological composition of claim 1, wherein the lecithin component comprises phosphatidylcholine, phosphatidylethanolamine, phosphatidylinositol, YN100 Cadberry, synthetic phosphated diglyceride, or a combination thereof.

4. The dermatological composition of claim 1, wherein the lecithin component comprises a phospholipid having formula C₈H₁₇O₅NRR′, wherein R and R′ are fatty acids having formula CH₃(CH₂)ₙCOO, with n equal to between 4 and 22.

5. The dermatological composition of claim 1, wherein the lecithin component comprises 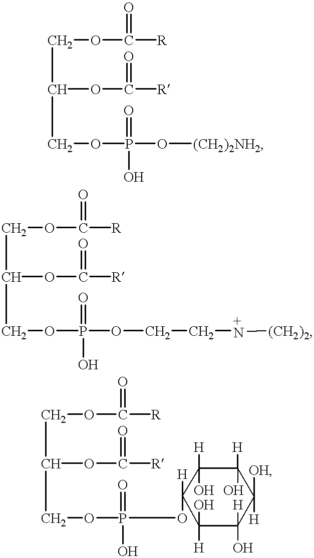
or a combination thereof, wherein R and R′ are fatty acids having the formula CH₃(CH₂)ₙCOOH, with n equal to between 4 and 22.

6. The dermatological composition of claim 1, wherein the additive component comprises fragrance, wax, fatty acid, oil, moisturizer, vitamin, ultraviolet light blocker, germicide, humectant, cleaning agent, colloidal meal, nutritional supplement, chelated mineral, herbal extract, essential fatty acid, drug, phytochemical, amino acid, disintegrant, or a combination thereof.

7. The dermatological composition of claim 6, wherein the oil comprises jojoba oil, emu oil, almond oil, macadamia nut oil, borage oil, black current oil, evening primrose oil, high oleic sunflower oil, vegetable oil, or a combination thereof.

8. The dermatological composition of claim 6, wherein the vitamin comprises tocotrienol, tocopherol, ascorbic acid, or a combination thereof.

9. The dermatological composition of claim 6, wherein the phytochemical comprises polyphenols, sterols, stanols, or a combination thereof.

10. The dermatological composition of claim 1, wherein said dermatological composition is adapted to improve psoriasis and said additive component comprises tocotrienols, emu oil, borage oil, evening primrose oil, omega 3 oils, or a combination thereof.

11. The dermatological composition of claim 1, wherein said dermatological composition is adapted to reduce the effects of UV exposure to the skin and said additive component comprises tocopherol, tannic acid, epigallocatechin, or a combination thereof.

12. A process for producing a dermatological composition comprising.
a) combining lecithin, water, water-soluble agents and polyethylene glycol to produce an aqueous mixture;
b) combining lipid soluble ingredients under constant mixing to produce a lipid soluble combination;
c) adding said lipid soluble combination to said aqueous mixture to produce a composition body;
d) adding at least one bactericide to said composition body;
e) cooling said composition body;
f) reheating said composition body; and
g) adding at least one fragrance to said composition body.

13. The process of claim 12, wherein said step of combining to produce the aqueous mixture occurs at a temperature of between about 65° C. and about 70° C.

14. The process of claim 12, wherein said step of combining to produce the lipid soluble combination occurs at a temperature of between about 70° C. and about 80° C.

15. The process of claim 12, wherein said step of reheating said composition body comprises reheating the composition body to a temperature of about 70° C.

16. A method for repairing damaged skin comprising utilizing a dermatological composition in a therapeutically effective manner, said dermatological composition comprising a lecithin component and dermatological nutrients, said lecithin component comprising between about 10% and about 30% by weight of the total weight of the dermatological composition.

17. The method of claim 16, wherein said dermatological nutrients comprise at least one vitamin.

18. The method of claim 17, wherein said at least one vitamin comprises tocotrienol, tocopherol, ascorbic acid, or a combination thereof.

19. A method for maintaining integrity of skin cell membranes comprising utilizing a dermatological composition in a therapeutically effective manner, said dermatological composition comprising a lecithin component and at least one phytochemical, said lecithin component comprising between about 10% and about 30% by weight of the total weight of the dermatological composition, said lecithin component having an acetone insoluble content equal to at least about 90% and a density from about 0.93 to about 1.2.

20. The method of claim 19, wherein said at least one phytochemical comprises polyphenols, sterols, stanols, or a combination thereof.

21. A method for stabilizing liposomes formed when lecithin phospholipids of a dermatological composition are hydrated comprising utilizing the dermatological composition in a therapeutically effective manner, said dermatological composition comprising a lecithin component and at least one phytochemical, said lecithin component comprising between about 10% and about 30% by weight of the total weight of the dermatological composition, said lecithin component having an acetone insoluble content equal to at least about 90% and a density from about 0.95 to about 1.2.

22. The method of claim 21, wherein said at least one phytochemical comprises polyphenols, sterols, stanols, or a combination thereof.

23. A method for improving psoriasis comprising utilizing a dermatological composition in a therapeutically effective manner, said dermatological composition comprising a lecithin component and at least one secondary active agent, said lecithin component comprising between about 10% and about 30% by weight of the total weight of the dermatological composition, said lecithin component having an acetone insoluble content equal to at least about 90% and a density from about 0.95 to about 1.2.

24. The method of claim 23, wherein said lecithin component comprises phospholipids in an amount equal to at least about 20% by weight of the total weight of the dermatological composition.

25. The method of claim 23, wherein said at least one secondary active agent comprises tocotrienols, emu oil, borage oil, evening primrose oil, omega 3 oils, or a combination thereof.

26. A method for reducing the effects of ultraviolet exposure comprising utilizing a dermatological composition in a therapeutically effective manner, said dermatological composition comprising a lecithin component and at least one secondary active agent, said lecithin component comprising between about 10% and about 30% by weight of the total weight of the dermatological composition, said lecithin component having an acetone insoluble content equal to at least about 90% and a density from about 0.95 to about 1.2.

27. The method of claim 26, wherein said lecithin component comprises phospholipids in an amount equal to at least about 20% by weight of the total weight of the dermatological composition.

28. The method of claim 26, wherein said at least one secondary active agent comprises tocopherol, tannic acid, epigallocatechin, or a combination thereof.
